(19) United States
(12) Patent Application Publication
(10) Pub. No.: US 2005/0137443 A1
(43) Pub. Date: Jun. 23, 2005

(54) Regenerative removal of trace carbon monoxide

(76) Inventors: Jayant K. Gorawara, Buffalo Grove, IL (US); Henry Rastelli, Gurnee, IL (US)

Correspondence Address: JOHN G TOLOMEI, PATENT DEPARTMENT;UOP LLC, 25 EAST ALGONQUIN ROAD, P O BOX 5017, DES PLAINES, IL, 60017-5017, US

(21) Appl. No.: 10/741,739

(22) Filed: Dec. 19, 2003

(51) Int. Cl. C07C 7/12

(52) U.S. Cl. 585/823

(57) ABSTRACT

By the present invention, a process is provided to use a modified clinoptilolite adsorbent suitable for the separation of carbon monoxide from hydrogen and hydrocarbon streams without adsorbing hydrocarbons such as paraffins and olefins. In typical applications in platforming units within refineries, these hydrogen streams contain from 5 to 20 parts per million of carbon monoxide. In other applications the level of carbon monoxide may be higher. The separation of carbon monoxide from the hydrogen stream is achieved by using a clinoptilolite molecular sieve that has been ion-exchanged with at least one cation selected from lithium, sodium, potassium, calcium, barium, and magnesium.

### FIELD OF THE INVENTION

This invention relates to processes for the purification of hydrocarbon and hydrogen containing streams. More specifically, this invention relates to processes for the use of adsorbents including modified clinoptilolites for the removal of carbon monoxide from said streams. The clinoptilolites may be natural or synthetic clinoptilolites which have been modified by ion-exchange with one or more metal cations.

### BACKGROUND OF THE INVENTION

Processes exist for separating feed streams containing molecules having differing sizes and shapes by contacting the feed stream with an adsorbent into which one component of the feed stream to be separated is more strongly adsorbed by the adsorbent than the other. The more strongly adsorbed component is preferentially adsorbed by the adsorbent to provide a first product stream which is enriched in the weakly or non-adsorbed component as compared with the feed stream. After the adsorbent is loaded to a desired extent with the adsorbed component, the conditions of the adsorbent are varied, e.g., typically either the temperature of or the pressure upon the adsorbent is altered, so that the adsorbed component can be desorbed, thereby producing a second product stream which is enriched in the adsorbed component as compared with the feed stream.

Important factors in such processes include the capacity of the molecular sieve for the more strongly adsorbable components and the selectivity of the molecular sieve (i.e., the ratio in which the components to be separated are adsorbed). In many such processes, zeolites are the preferred adsorbents because of their high adsorption capacity at low partial pressures of adsorbates and, when chosen so that their pores are of an appropriate size and shape to provide a high selectivity in concentrating the adsorbed species.

Often the zeolites used in the separation of gaseous mixtures are synthetic zeolites. Although natural zeolites are readily available at low cost, natural zeolites are often not favored as adsorbents because it has been felt that the natural zeolites are not sufficiently consistent in composition to be useful as adsorbents in such processes. However, there are relatively few synthetic zeolites with pore sizes in the range of about 3 to 4 Å, which is the pore size range of interest for a number of gaseous separations.

Clinoptilolites (frequently referred to as “clino” hereinafter) are a well-known class of natural zeolites which have occasionally been proposed for the separation of gaseous mixtures, usually light gases such as hydrogen, nitrogen, oxygen, argon, or methane.

U.S. Pat. No. 5,116,793 describes a process for ion exchange of clinoptilolites with metal cations such as lithium, sodium, potassium, calcium, magnesium, barium, strontium, zinc, copper, cobalt, iron and manganese. This patent is incorporated herein in its entirety.

In U.S. Pat. No. 4,935,580 ion exchanged clinoptilolites are disclosed that remove traces of carbon dioxide and water from streams of hydrocarbons.

U.S. Pat. No. 5,019,667 discloses the use of modified clinoptilolite wherein at least about 40% of the ion-exchangeable cations in the clinoptilolite comprise any one or more of lithium, potassium, calcium, magnesium, barium, strontium, zinc, copper, cobalt, iron and manganese cations. This clinoptilolite is used to remove ammonia from hydrocarbon streams.

Accordingly, processes are sought which can separate carbon monoxide from hydrogen and hydrocarbons, without removing hydrogen and hydrocarbons such as methane, ethane, ethylene, propane and propylene, by adsorption using adsorbents. Modified clinoptilolite adsorbents have been found to achieve this goal as have titanium silicates and natural zeolites including mordenite having pore sizes smaller than a 4 Å product (and larger than a 3 Å product). Moreover, processes for the production of the modified clinoptilolite adsorbents are sought.

The catalyst reforming unit is an integral part of and also is a supplier of a refinery's hydrogen production. With the advent of low pressure, high severity catalytic reforming units, the presence of carbon monoxide (CO) in the net hydrogen gas from reforming units is becoming more prevalent. Some of the processes, such as paraffin isomerization units that use this hydrogen, have catalysts that are very sensitive to CO (as well as to other oxygenates) and if the carbon monoxide is not removed the catalyst is poisoned. One of the methods currently used for removing carbon monoxide is to employ a methanator, to react hydrogen with carbon monoxide, producing methane and water. While the methanator is considered the primary tool to address the contamination problem this is very capital intensive as well as consuming energy and using up hydrogen. While some consideration has been made to using adsorbents to remove the carbon monoxide in such processes, it was previously believed that the adsorbents that would remove carbon monoxide will coadsorb hydrocarbons such as ethylene which exist in significantly higher concentrations, thereby greatly diminishing the capacity for removal of carbon monoxide.

### SUMMARY OF THE INVENTION

By the present invention, a process is provided to use an adsorbent, and preferably a modified clinoptilolite adsorbent, suitable for the separation of carbon monoxide from hydrocarbon and hydrogen containing streams. In typical applications in platforming units within refineries, these hydrocarbon and hydrogen containing streams contain from 5 to 20 parts per million of carbon monoxide. In other applications the level of carbon monoxide may be higher. For example, there may be streams with as much as 1% carbon monoxide to be purified. These hydrocarbon and hydrogen containing streams may further contain hydrocarbons, including ethane and ethylene. The separation of carbon monoxide from the stream is achieved by using a clinoptilolite molecular sieve that has been ion-exchanged with at least one cation selected from lithium, sodium, potassium, calcium, barium, and magnesium. Preferably, the clinoptilolite adsorbent is ion-exchanged to an extent such that at least about 60% of the total cations in the clinoptilolite are occupied by one or more of the listed cations. The process removes at least 50% and preferably at least 90% of the carbon monoxide from such hydrogen and hydrocarbon containing streams, without removing hydrocarbons such as ethylene.

The present invention provides for the use of an adsorbent to remove carbon monoxide, including the use of a modified clinoptilolite wherein at least about 40% of the ion-exchangeable cations in the clinoptilolite comprise any one or more of lithium, potassium, calcium, sodium, magnesium, or barium cations. One process by which the modified clinoptilolite is made is by subjecting a natural occurring clinoptilolite to ion-exchange with a solution containing sodium cations until at least about 40% of the ion-exchangeable non-sodium cations in the clinoptilolite have been replaced by sodium cations, thereby producing a sodium clinoptilolite, and thereafter subjecting said sodium clinoptilolite to ion-exchange with a solution containing any one or more of lithium, sodium, potassium, calcium, barium, and magnesium cations.

In another process, the modified clinoptilolite is made by directly subjecting a clinoptilolite to ion-exchange with a solution containing any one or more of lithium, sodium, potassium, calcium, barium, and magnesium cations. The preferred modified clinoptilolite is ion-exchanged with calcium. Other adsorbents may also be used that have a pore size that is intermediate between the pore size of zeolites 3 Å and 4 Å such as titanium silicates which can be tailored to having specific pore sizes and shapes.

In yet another process, the present invention comprises a process for the production of high purity hydrogen from a catalytic reformer which process comprises the steps of passing at least a portion of a hydrogen gas stream produced in the catalytic reformer and comprising carbon monoxide to a adsorbent bed containing an adsorbent having an effective pore size and shape that excludes hydrocarbon molecules and is large enough to adsorb carbon monoxide molecules. At least a portion of the hydrogen gas stream having a reduced concentration of carbon monoxide is passed to a catalytic hydrocarbon conversion process requiring hydrogen containing low levels of carbon monoxide.

The catalytic reforming unit is an integral part of and supplier of a refinery's hydrogen production. With the advent of low pressure catalytic reforming processes, the presence of carbon monoxide in the net hydrogen gas is becoming more prevalent. Some of the processes, such as paraffin isomerization units, that use this hydrogen have catalysts that are very sensitive to this CO (as well as other oxygenates). The current method of removing this poison is to employ a methanator, which is capital intensive while also consuming utilities, including hydrogen. A thermal swing adsorption unit is frequently used to dry the hydrogen. The judicious use of an adsorbent such as a clino (sodium or calcium forms) to exclude the C₂⁺ hydrocarbons in the hydrogen stream can allow the adsorption of CO. An existing swing bed adsorption system for dehydration can be used in most cases while modifying the cycle time and adsorbents currently used.

### DETAILED DESCRIPTION OF THE INVENTION

A thermal swing adsorption system is used to dry the hydrogen in a paraffin isomerization unit. The judicious use of an adsorbent such as a clinoptilolite (sodium or calcium forms) to exclude the C₂⁺ hydrocarbons in the hydrogen stream can allow the adsorption of CO. An existing thermal swing adsorption system for dehydration can be used for CO removal in most cases. Using the existing thermal swing hydrogen dryers in the paraffin isomerization (Butamer™ and Penex™) units one could modify the cycle and use a compound bed of adsorbents in the existing vessels for simultaneous removal of water and CO. In the past, it was not considered practical to use thermal swing process for CO removal due to co-adsorption of heavier hydrocarbons from the process stream which severely limited the adsorbent's CO capacity. This limitation is addressed by using an adsorbent with a pore size and pore opening shape that excludes the hydrocarbon species that would normally co-adsorb.

The invention provides lower capital and operating costs; in many cases existing vessels and equipment can be used to enhance performance by removing a severe catalyst poison (in this case for the paraffin isomerization unit).

The hydrogen dryers designed for most paraffin isomerization units can be used for both dehydration and carbon monoxide removal. These thermal swing units therefore have the capacity for contaminant removal in addition to dehydration. Prior to the present invention, it was not believed that trace CO could be effectively removed from this hydrogen stream using a thermal swing process due to very low expected capacity a consequence of co-adsorption of C₂⁺ hydrocarbons. The CO concentration in the net hydrogen stream from the catalytic reforming unit is typically in the range of 5 to 20 ppm(m). This level of contaminant can be removed by using a compound bed of adsorbent for water removal followed by an adsorbent for CO removal. One can thus expect to enhance the performance of the paraffin isomerization catalyst without the expensive addition of a methanator. While trace levels of CO can be adsorbed from hydrogen alone by many adsorbents, it was expected prior to the present invention that in the presence of hydrocarbons, hydrocarbon co-adsorption would be expected to diminish its capacity considerably for the typical adsorbents that would be thermally regenerated.

It is known that the adsorption properties of many zeolites, and hence their ability to separate gaseous mixtures, can be varied by incorporating various metal cations into the zeolites, typically by ion-exchange or impregnation. Thus, potassium A is commonly referred to as having an effective pore diameter of 3 Å and calcium A similarly is referred to as having an effective pore diameter of 5 Å. The term “effective pore diameter” is used in order to functionally define the pore size of a molecular sieve in terms of what molecules it can adsorb rather than actual dimensions which are often irregular and non-circular, e.g. elliptical. D. W. Breck, in ZEOLITE MOLECULAR SIEVES, John Wiley and Sons (1974), hereby incorporated by reference, describes effective pore diameters at pages 633 to 641.

In most cases, the changes in the adsorption properties of zeolites following ion-exchange are consistent with a physical blocking of the pore opening by the cation introduced; in general, in any given zeolite, the larger the radius of the ion introduced, the smaller the effective pore diameter of the treated zeolite (for example, the pore diameter of potassium A zeolite is smaller than that of calcium A zeolite), as measured by the size of the molecules which can be adsorbed into the zeolite.

This is not the case, however, with clinoptilolites which demonstrate an unpredictable relationship that is not a simple function of the ionic radii of the cation introduced, i.e., pore blocking. For example, unlike the above-described calcium and potassium ion-exchanged forms of zeolite A, clinoptilolite produces the opposite effect with these two cations. That is, potassium cations, which are larger than calcium cations, provide a clinoptilolite having a larger effective pore diameter than calcium ion-exchanged clinoptilolite. In fact, a calcium ion-exchanged clinoptilolite with a calcium content equivalent to 90% of its ion-exchange capacity defined by its aluminum content essentially excludes both nitrogen and methane. On the other hand, a potassium ion-exchanged clinoptilolite with a potassium content equivalent to 95% of its ion-exchange capacity adsorbs both nitrogen and methane rapidly. Here, the clinoptilolite containing the cation with the larger ionic radii, i.e., potassium, has a larger pore than the clinoptilolite containing the cation with the smaller ionic radii, i.e., calcium.

The clinoptilolites used in the process of the present invention may be natural or synthetic clinoptilolites. Synthetic clinoptilolites are not easily synthesized, as noted in ZEOLITE MOLECULAR SIEVES, supra at pg 260, and accordingly natural clinoptilolites are preferred. However, natural clinoptilolites are variable in composition and chemical analysis shows that the cations in clinoptilolites samples from various mines vary widely. Moreover, natural clinoptilolites frequently contain substantial amounts of impurities, especially soluble silicates, which may cause difficulties in the aggregation or pelletization of the clinoptilolite (discussed in more detail below), or may cause undesirable side-effects which would inhibit practicing the present invention. In some applications, the mesh form of the adsorbent is preferred over the pelletized form of it.

In accordance with the present invention, it is required that the clinoptilolites be modified by ion-exchange with at least one metal cation in order to establish the appropriate pore size and shape to perform the separation and to establish compositional uniformity. Among the cations which can usefully be ion-exchanged into clinoptilolites are lithium, potassium, magnesium, calcium, sodium and barium cations. Thus, any cation which has the desired effect on pore size can be used for ion-exchange. Moreover, the choice of a particular cation can be dependent on the characteristics of the starting material. Desirably, the ion-exchange is continued until the final ion exchanged clino product contains greater than 40% of the desired cations. The preferred metal cations for treatment of the clinoptilolites used in the process of the present invention are calcium, magnesium, and barium cations, with calcium being especially preferred. When calcium is used as the ion-exchange metal cation, it is preferred that the ion-exchange be continued until at least about 60% of the total cations in the clinoptilolite are calcium cations.

It should be noted that the ion-exchanging can be done in two or more steps. For example, ion-exchanging can be employed to provide a compositionally uniform starting material that is suitable for additional ion-exchanging for pore size tailoring. Thus, additional ion-exchanging can be employed in order to compensate for inherent differences in the naturally occurring raw material thereby enhancing the performance for separating carbon monoxide from hydrocarbons and hydrogen.

Since clinoptilolite is a natural material, the particle sizes of the commercial product varies, and the particle size of the clinoptilolite may effect the speed and completeness of the ion-exchange reaction.

Techniques for the ion-exchange of zeolites such as clinoptilolite are well-known to those skilled in the molecular sieve art, and hence will not be described in detail herein. In the ion-exchange, the cation is conveniently present in the solution in the form of its water soluble salt form. It is desirable that the ion-exchange be continued until at least about 40%, and preferably at least about 50%, of the cation content is the desired cation. It is convenient to continue the ion-exchange until no further amount of the desired cation can easily be introduced into the clinoptilolite. To secure maximum replacement of the original clinoptilolite cations, it is recommended that the ion-exchange be conducted using a solution containing a quantity of the cation to be introduced which is from about 2 to about 100 times the ion-exchange capacity of the clinoptilolite. Typically, the ion-exchange solution will contain from about 0.1 to about 5 moles per liter of the cation, and will be contacted with the original clinoptilolite for at least about 1 hour. The ion-exchange may be conducted at ambient temperature, although in many cases carrying out the ion-exchange at elevated temperatures, usually less than 100° C., accelerates the ion-exchange process.

Since clinoptilolite is a natural material of variable composition, the cations present in the raw clinoptilolite vary, although typically the cations include a major proportion of alkali metals. It is typically found that, even after the most exhaustive ion-exchange, a proportion of the original clinoptilolite cations, i.e., from about 5 to 15 wt-% cannot be replaced by other cations. However, the presence of this small proportion of the original clinoptilolite cations does not interfere with the use of the ion-exchanged clinoptilolites in the process of the present invention.

As noted above, any of the modified clinoptilolites used in the present invention can be prepared directly by ion-exchange of natural clinoptilolite with the appropriate cation. However, in practice such direct ion-exchange may not be the most economical or practical technique. Being natural minerals, clinoptilolites are variable in composition and frequently contain substantial amounts of impurities, especially soluble silicates. To ensure as complete an ion-exchange as possible, and also to remove impurities, it is desirable to effect the ion-exchange of the clinoptilolite using a large excess of the cation which it is desired to introduce. However, if, for example, a large excess of barium is used in such an ion-exchange, the disposal and/or recovery of barium from the used ion-exchange solution presents a difficult environmental problem, in view of the limitations on release of poisonous barium salts into the environment. Furthermore, some impurities, including some silicates, which are removed in a sodium ion-exchange are not removed in a barium ion-exchange because the relevant barium compounds are much less soluble than their sodium counterparts.

When the clinoptilolites of the present invention are to be used in industrial adsorbers, it may be preferred to aggregate (pelletize) the modified clinoptilolite to control the macropore diffusion, or in an industrial size adsorption column, pulverulent clinoptilolite may compact, thereby blocking, or at least significantly reducing flow through, the column. Those skilled in molecular sieve technology are aware of conventional techniques for aggregating molecular sieves; such techniques usually involve mixing the molecular sieve with a binder, which is typically a clay, forming the mixture into an aggregate, typically by extrusion or bead formation, and heating the formed molecular sieve/clay mixture to a temperature of about 500° to 700° C. to convert the green aggregate into one which is resistant to crushing.

The binders used to aggregate the clinoptilolites may include clays, silicas, aluminas, metal oxides and mixtures thereof. In addition, the clinoptilolites may be formed with materials such as silica, alumina, silica-alumina, silica-magnesia, silica-zirconia, silica-thoria, silica-berylia, and silica-titania, as well as ternary compositions, such as silica-alumina-thoria, silica-alumina-zirconia and clays present as binders. The relative proportions of the above materials and the clinoptilolites may vary widely with the clinoptilolite content ranging between about 1 and about 99, preferably between about 60 and 95, percent by weight of the composite. Where the clinoptilolite is to be formed into aggregates prior to use, such aggregates are desirably about 1 to about 4 mm in diameter.

To avoid the aforementioned difficulties, it is sometimes preferred to produce modified clinoptilolites other than sodium clinoptilolite by first subjecting raw clinoptilolite to a sodium ion-exchange, aggregating the sodium clinoptilolite thus produced, and then effecting a second ion-exchange on the aggregated material to introduce the desired non-sodium cations.

Before being used in the processes of the present invention, the clinoptilolites need to be activated by calcining, i.e., heating. If the clinoptilolite is aggregated as discussed above, the heat required for aggregation will normally be sufficient to effect activation also, so that no further heating is required. If, however, the clinoptilolite is not to be aggregated, a separate activation step will usually be required. Moreover, if the ore is used directly or ion-exchange is conducted after the aggregation, a separated activation step usually will be required. Clinoptilolites can be activated by heating in air, inert atmosphere, or vacuum to a temperature and for a time sufficient to cause the clinoptilolite to become activated. The term “activated” is used herein to describe an adsorbent having reduced water content relative to being in equilibrium with atmospheric air. Typical activation conditions include a temperature of 100° to 700° C. and a time of 30 minutes to 20 hours which is sufficient to reduce the water content of clinoptilolite to about 0.2 to 2 wt-%. Preferably the clinoptilolites are activated by heating in an air or nitrogen purge steam or in vacuum at approximately 200° to 350° C. for a suitable period of time. The temperature needed for activation of any particular specimen of clinoptilolite can be easily determined by routine empirical tests where typical adsorption properties such as absolute loadings or adsorption rates are measured for samples activated at various temperatures.

Although ion-exchange of clinoptilolite does produce a modified clinoptilolite having a consistent pore size, the exact pore size depends not only upon the metal cation(s) exchanged but also upon the thermal treatment of the product following ion-exchange. In general, there is a tendency for the pore size of the modified clinoptilolites of this invention to decrease with exposure to increasing temperature. Accordingly, in selecting an activation temperature for the modified clinoptilolites, care should be taken not to heat modified clinoptilolites to temperatures which cause reductions in pore size so severe as to adversely affect the performance of the modified clinoptilolite in the process of the present invention, i.e., higher than 700° C. Although the behavior of the modified clinoptilolites on exposure to heat does limit the activation temperatures which can be employed, the thermal reduction in pore size does offer the possibility of “fine tuning” the pore size of a modified clinoptilolite to optimize its performance in the process of the present invention.

The process of the present invention is primarily intended for removal of traces of carbon monoxide from hydrogen and hydrocarbon streams where the presence of even a few parts per million of carbon monoxide can be undesirable.

Since these types of processes involve the separation of minor amounts of carbon monoxide from much larger amounts of hydrogen and hydrocarbon streams, they may be effected in the conventional manner by simply passing the hydrogen stream through a bed of the clinoptilolite, which is normally in aggregate form during an adsorption step. As the adsorption step continues, there develops in the bed a so-called “front” between the clinoptilolite loaded with carbon monoxide and clinoptilolite not so loaded, and this front moves through the bed in the direction of gas flow. Preferably, the temperature during the adsorption step is maintained between about −15° to +100° C. Before the front reaches the downstream end of the bed (which would allow impure hydrogen gas to leave the bed), the bed is preferably regenerated by cutting off the flow of hydrogen gas and passing through the bed a purge gas which causes desorption of the carbon monoxide from the bed. In industrial practice, the purge gas is typically natural gas or vaporized isomerate product, heated to a temperature in the range of 100° to 350° C., and such a purge gas is also satisfactory in the processes of the present invention. It is also important to note that other adsorption cycles such as pressure swing or purge cycles can be employed. Such cycles form no critical part of the present invention, are well known to those skilled in the art, and accordingly, will not be further discussed herein.

The following Examples are given, though by way of illustration only, to show preferred processes of the present invention. All adsorption measurements are at 23° C. unless otherwise stated. Furthermore, all separation factors given in the form based upon the data in the examples, it was concluded that calcium exchanged clinoptilolite is the best candidate tested to date for CO removal from hydrogen net gas.

### EXAMPLES

### Example 1

The modified clinoptilolite was made in accordance with the following procedure:

First, determine the amount of salt solution needed through the following steps:

Select the clinoptilolite of interest, and estimate its formula weight from the moles and molecular weights of each oxide species present. Then, determine the equivalents per gram of active sample for each of the exchangeable cations present, and total the values. Calculate the amount of salt and solution stoichiometrically required to displace all of the cations (if total exchange is desired) in the active material. Typically, we multiply these values by four to compensate for imperfections in the sample and exchange conditions. The molarity of the salt solution has been limited to 0.4, or less, which is favorable for most exchanges (but not for all).

Make the exchange salt solution, and adjust its pH as follows: Measure and record the actual amount of salt used. Add the salt to a graduated bucket (precision is not critical when operating in excess), and add de-ionized water to the appropriate mark. If necessary, use a carboy for large volumes of solution. Prepare a solution of about 10 wt-% base (example: Ca(OH)₂ for a solution of CaCl₂) in water to adjust the pH of the salt solution to a pH of 9.9 to 10.2. This is favorable to most exchanges. Add about 0.3 to 0.5 mL aliquots of the base solution to the salt solution, and measure the salt solution pH with pH paper after each addition. Record the amount used for future reference.

Prepare the wash solution, and adjust its pH as follows: The wash solution uses the same salt as the exchange solution, but is very dilute (example: if the exchange solution is 0.2 M, then the wash would be 0.2M/20, or 0.01M). Measure the amount of salt needed, and record its mass. Complete the solution preparation and pH adjustment in the same manner as the exchange solution.

Load the exchange column as follows: With a lightly silicone greased O-ring in place, attach the bottom Teflon fitting to the column. Through the top of the column, insert one piece of stainless steel mesh to cover the orifice in the bottom fitting. On top of the mesh, add about 0.2 kg (0.5 pound) of 6 mm glass beads (about one-half of a bottle) to serve as a preheat section. Add three pieces of stainless steel mesh to separate the adsorbent sample from the beads. Weigh and record the actual amount of clinoptilolite sample, and add it to the column. Place two pieces of stainless steel mesh on top of the sample, and fill the column close to the top with 6 mm glass beads (to reduce dead volume). Install the top O-ring (lightly greased) and fitting. If conducting a heated exchange, as is typical, turn on the water bath and set the dial at about 88° C., or lower as desired.

Complete the exchange as follows: Start pumping the salt solution at about 33 mL/minute. Record the start time and the measured flow rate. Place the effluent tube end into a waste bucket or carboy. Check the water bath and pump occasionally to ensure all is operating properly. When the exchange solution is completely dispensed, immediately start pumping the wash solution at the same rate and temperature. When the pumping of the wash solution is completed, connect the effluent tube to house air, and place the column inlet tube end into the waste container. While maintaining the temperature of the column, allow house air to pass through the column at a reasonable rate to dry the clinoptilolite sample (1 to 3 hours). Turn off the water bath to allow the column to cool, but maintain the house air flow to aid in cooling. When the column is cool, carefully remove the sample through the bottom of the column.

Activate the sample and submit it for analysis as follows: For gentle removal of water, this “pre-activation” with house air can be used:

| Ramp (hrs) | Temp. (° C.) | Hold (hrs) |
|---|---|---|
| 0.5 | 50 | 0.5 |
| 1.5 | 100 | 5 |
| 1.5 | 150 | 4 |
| 1.5 | 200 | 2 |
| 1.5 | 250 | 2 |
| 1.0 | 25 | 2 |

Finally, vacuum activate the sample for about three hours at 350° C., allow it to cool to around 80° C., bottle, and submit a portion for analytical tests (usually LOI and ICP).

### Example 2

In initial testing on various zeolite materials, only barium exchanged clinoptilolite (clino) mesh exhibited enough adsorption capacity for CO at low partial pressures to be of interest in purification applications. A starting sample of modified clino was produced by sodium exchanging the fresh clino ore. This sodium exchanged clino was used as the starting material to produce the ion-exchanged forms of potassium, lithium, and calcium to find an optimum adsorbent for CO which still excludes hydrocarbons. The samples were sent for chemical analysis to verify the extent of the ion exchange as shown in Table 1 below.

The materials were then tested for CO adsorption. After the samples were thoroughly activated, CO was adsorbed at 6 torr partial pressure for 3 hours. Then the CO pressure was increased to 46 torr and adsorbed for 2 hours. (Table 2) Equilibrium was apparently achieved at both conditions. The samples were then subjected to vacuum overnight at 350° C. to reactivate them. The next day, they were tested for hydrocarbon co-adsorption. First they were tested with propane at 250 torr and 21° C. (Table 3) and, after another activation, ethylene at 700 torr and 21° C. (Table 4).

Four samples were previously made materials and were tested as is. Two were samples of barium clinoptilolite. The CO data for these two were almost identical which verifies the reproducibility of the CO McBain testing technique that was used. The Mg Clino was a magnesium exchanged clino. The sodium exchanged clino is a mesh product and the current commercial product sold into the hydrocarbon processing business. Three ion-exchange forms were made from this material: the calcium, lithium and potassium forms of clino.

TABLE 1
Ion-Exchange Forms of Clino

| | Sample No. | | | | | |
|---|---|---|---|---|---|---|
| | 1 | 2 | 3 | 4 | 5 | 6 |
| | Oxide ID | | | | | |
| | XO/Al₂O₃ | XO/Al₂O₃ | XO/Al₂O₃ | XO/Al₂O₃ | XO/Al₂O₃ | XO/Al₂O₃ |
| | Major Cation | | | | | |
| | Mg | Ba | Na | Ca | Li | K |
| SiO₂ | 10.39 | 10.59 | 10.35 | 10.35 | 10.48 | 10.50 |
| TiO₂ | 0.01 | 0.01 | 0.01 | 0.01 | 0.01 | 0.01 |
| Fe₂O₃ | 0.05 | 0.04 | 0.05 | 0.05 | 0.05 | 0.04 |
| Al₂O₃ | 1.00 | 1.00 | 1.00 | 1.00 | 1.00 | 1.00 |
| BaO | 0.00 | 0.72 | 0.00 | 0.00 | 0.00 | 0.00 |
| MgO | 0.55 | 0.08 | 0.12 | 0.12 | 0.09 | 0.08 |
| CaO | 0.18 | 0.04 | 0.35 | 0.66 | 0.20 | 0.03 |
| Na₂O | 0.34 | 0.06 | 0.64 | 0.27 | 0.14 | 0.07 |
| K₂O | 0.14 | 0.14 | 0.28 | 0.26 | 0.24 | 0.88 |
| Li₂O | 0.01 | 0.01 | 0.01 | 0.01 | 0.56 | 0.01 |
| Tot. Cations | 1.22 | 1.05 | 1.40 | 1.32 | 1.24 | 1.07 |
| Tot (Na + K) | 0.48 | 0.20 | 0.92 | 0.53 | 0.39 | 0.95 |
| Cat sites* | 0.44 | 0.72 | | 0.39 | 0.54 | 0.85 |
| Delta cation | 0.43 | 0.72 | | 0.31 | 0.55 | 0.88 |
| Delta/sites | 98% | 100% | | 79% | 102% | 104% |

*Difference between (Na + K) at start to (Na + K) at finish, estimate only since some of the Mg and Ca should be included but the quantity is indeterminable

For CO adsorption, the following order was found at 7 torr CO partial pressure (Table 2): The results showed that Ba Clino>Ca Clino>Li Clino>Na Clino in pellet form>Mg Clino>Na Clino=K clino.

TABLE 2
McBain Result

| | Sample # | Loading, wt- % | | | | | | |
|---|---|---|---|---|---|---|---|---|
| | Ads. Press. (VG), Torr | 6.0E+00 | 5.5E+00 | 6.0E+00 | 6.5E+00 | N/A | N/A | N/A |
| | Ads. Press. (PV), Torr | 6 | 6 | 7 | 7 | 46 | 47 | 47 |
| | Gas Phase | CO | CO | CO | CO | CO | CO | CO |
| # | Sample #/Ads. Time, minutes | 10 | 50 | 120 | 180 | 210 | 280 | 340 |
| 1 | Na Clino Mesh | 0.09 | 0.11 | 0.16 | 0.16 | 0.55 | 0.68 | 0.72 |
| 2 | Ba Clino Mesh | 1.13 | 1.28 | 1.31 | 1.31 | 2.46 | 2.53 | 2.52 |
| 3 | Mg Clino Mesh | 0.18 | 0.19 | 0.25 | 0.26 | 0.56 | 0.67 | 0.65 |
| 4 | Ca Clino Mesh | 0.45 | 0.77 | 0.85 | 0.91 | 1.35 | 1.45 | 1.47 |
| 5 | Na Clino Pellets | 0.44 | 0.44 | 0.47 | 0.46 | 0.98 | 1.01 | 1.00 |
| 6 | Li Clino Mesh | 0.40 | 0.45 | 0.49 | 0.48 | 1.30 | 1.37 | 1.39 |
| 7 | K Clino Mesh | 0.14 | 0.12 | 0.14 | 0.16 | 0.56 | 0.57 | 0.56 |
| 8 | Ba Clino Mesh | 0.89 | 1.34 | 1.37 | 1.37 | 2.59 | 2.66 | 2.66 |

For propane exclusion (less is better) the following was found (Table 3): A summary of the results shown in Table 3 is that adsorption by Mg Clino=Ca clino=Na clino=K clino=Li clino<Ba Clino<Na clino pellets.

TABLE 3
McBain Result

| | Sample # | Activated | Wt. Loss, | Loading, % | Loading, % | Loading, % |
|---|---|---|---|---|---|---|
| | | Weight | %, Dry | | | |
| | | at Start | Base | | | |
| | Ads. Press. (VG), | 4.0E−03 | 4.0E−03 | 1.3E+01 | 1.3E+01 | 1.3E+01 |
| | Torr | | | | | |
| | Ads. Press. (PV), Torr | −1 | −1 | 252 | 252 | 252 |
| | Gas Phase | | vac | C₃H₈ | C₃H₈ | C₃H₈ |
| | Sample #/Ads. | | | 30 | 45 | 60 |
| # | Time, minutes | | | | | |
| 1 | Na Clino M | 88.38 | 10.39 | 0.02 | 0.00 | 0.00 |
| 2 | Ba Clino M | 88.14 | 9.24 | 0.23 | 0.26 | 0.26 |
| 3 | Mg Clino M | 83.48 | 11.64 | −0.02 | −0.04 | −0.04 |
| 4 | Ca Clino M | 94.28 | 8.62 | −0.01 | −0.02 | −0.02 |
| 5 | Na Clino P | 86.81 | 11.97 | 0.33 | 0.35 | 0.35 |
| 6 | Li Clino M | 93.37 | 2.53 | 0.03 | 0.04 | 0.05 |
| 7 | K Clino M | 93.62 | 4.44 | 0.01 | 0.02 | 0.02 |
| 8 | Ba Clino M | 82.68 | 12.71 | 0.06 | 0.12 | 0.11 |

For ethylene exclusion (less is better) the following was found (Table 4): The results at 960 minutes showed that Na Clino=Ca Clino<Mg Clino <<K Clino<Li Clino<Ba Clino<Na Clino (P).

TABLE 4
McBain Result

| | Sample # | Activated | Wt. Loss, % | Loading, wt- % | | | |
|---|---|---|---|---|---|---|---|
| | | Weight | Dry Base | | | | |
| | | at Start | | | | | |
| | Ads. Press. (VG), | 4.0E−03 | 4.0E−03 | 2.6E+01 | 2.6E+01 | 2.6E+01 | 2.6E+01 |
| | Torr | | | | | | |
| | Ads. Press. (PV), | −1 | −1 | 700 | 699 | 698 | 695 |
| | Torr | | | | | | |
| | Gas Phase | | vac | C₂H₄ | C₂H₄ | C₂H₄ | C₂H₄ |
| | Sample #/Ads. | | | 30 | 45 | 60 | 960 |
| # | Time, minutes | | | | | | |
| 1 | Na Clino M | 88.40 | 10.36 | −0.07 | −0.08 | −0.07 | 0.11 |
| 2 | Ba Clino M | 88.15 | 9.22 | 3.97 | 4.27 | 4.34 | 5.14 |
| 3 | Mg Clino M | 83.49 | 11.63 | −0.05 | −0.02 | 0.00 | 0.40 |
| 4 | Ca Clino M | 94.30 | 8.60 | −0.06 | −0.06 | −0.02 | 0.16 |
| 5 | Na Clino P | 77.84 | 24.87 | 12.81 | 12.90 | 12.87 | 13.16 |
| 6 | Li Clino M | 93.38 | 2.52 | 0.78 | 0.99 | 1.11 | 2.84 |
| 7 | K Clino M | 93.65 | 4.41 | 0.19 | 0.29 | 0.33 | 1.38 |
| 8 | Ba Clino M | 82.68 | 12.71 | 3.74 | 4.06 | 4.25 | 5.24 |

Although in certain circumstances, other forms of clino such as sodium form will and do work well, for the particular application in H₂ purification, the calcium exchanged version of clino is the best candidate for removal of carbon monoxide, while adsorbing hydrocarbons.

### Example 3

Since calcium exchanged clino had the best combination of good CO loading with the least amount of hydrocarbon co-adsorption (propane, ethylene), further study was made at calcium exchanged forms of clino. Two different raw ores of clino were tested. Calcium exchanging the raw ore, without going through the sodium exchange to form a sodium exchanged ore first, is a significant cost reduction. Each ore was column exchanged. The chemical analysis of the starting ore and calcium exchanged form are depicted in Table 5.

TABLE 5
Ion Exchange Forms of Clino

| | Sample No. | | | |
|---|---|---|---|---|
| | 09674-24-16 | 32164-27-44 | 09674-24-09 | 32164-27-46 |
| | AMZ (TX-764) | | TSM-140 | |
| | | Ca Exch. | | Ca Exch. |
| | Base | TX-764 | Base | TSM-140 |
| | Oxide ID | | | |
| | XO/Al2O3 | XO/Al2O3 | XO/Al2O3 | XO/Al2O3 |
| | Location | | | |
| | Mobile | DP | DP | DP |
| SiO2 | 10.44 | 10.46 | 9.64 | 9.66 |
| TiO2 | 0.02 | 0.01 | 0.01 | 0.01 |
| Fe2O3 | 0.05 | 0.05 | 0.05 | 0.04 |
| Al2O3 | 1.00 | 1.00 | 1.00 | 1.00 |
| BaO | 0.00 | 0.00 | 0.00 | 0.00 |
| MgO | 0.13 | 0.11 | 0.13 | 0.13 |
| CaO | 0.40 | 0.76 | 0.24 | 0.52 |
| Na2O | 0.49 | 0.27 | 0.55 | 0.27 |
| K2O | 0.38 | 0.34 | 0.16 | 0.16 |
| Li2O | 0.00 | 0.01 | 0.00 | 0.00 |
| Tot. Cations | 1.39 | 1.50 | 1.09 | 1.07 |
| Tot (Na + K) | 0.87 | 0.62 | 0.72 | 0.43 |
| Cat sites* | | 0.25 | | 0.29 |
| Delta cation | | 0.37 | | 0.28 |
| Delta/sites | | 150% | | 96% |

*Difference between (Na + K) at start to (Na + K) at finish, estimate only since some of the Mg and Ca should be included but the quantity is indeterminable

Because of their unique layer-like structure, the clinos are easily pore closed. The higher the activation temperature, the more pore closure effect could be produced. Therefore, samples of the calcium exchanged forms of the clinos along with fresh clino ore were fired at 500° C. for 1 hour. This provided an indication as to the ease of producing a pore closure effect on these materials.

The materials were then tested for CO adsorption in the McBain-Bakr gravimetric adsorption apparatus. After the samples were thoroughly activated, CO was adsorbed at 6 torr partial pressure for 1.9 hours. Then the CO pressure was increased to 46 torr and adsorbed for 1 hour. (Table 6).

For CO adsorption at room temperature on fresh material, the following order was found (Table 6):

Ca TX-764>Ca EP-9174>Ca TSM-140>Ca TX-764*>>TSM-140>Ca TSM-140*>TX-764*=TX-764

TABLE 6
CO McBain Results

| | Sample # | | | | | |
|---|---|---|---|---|---|---|
| | Activated | Wt-% | | | | |
| | Weight | Loss | Loading | Loading | Loading | Loading |
| | At Start | dry base | WT % | WT % | WT % | WT % |
| | Ads. Press. (VG), Torr | | | | | |
| | 9.0E−03 | 8.5E−03 | | | | |
| | Ads. Press. (PV), Torr | | | | | |
| | | | 6 | 6 | 46 | 46 |
| | Ads. Time, Hr | | | | | |
| | | | CO @ | CO @ | CO @ | CO @ |
| Sample # | | | 1.0 hrs | 1.9 hrs | 0.5 hrs | 1.0 hrs |
| 07384-24-9 TSM-140 | 94.76 | 1.56 | 0.28 | 0.28 | 0.85 | 0.89 |
| 07384-24-16 TX-764 | 84.74 | 14.10 | 0.20 | 0.20 | 0.74 | 0.72 |
| 32164-027-44 Ca exchanged | 90.98 | 5.74 | 0.92 | 0.93 | 1.48 | 1.51 |
| TX-764 | | | | | | |
| 32164-027-46 Ca exchanged | 90.92 | 7.46 | 0.82 | 0.84 | 1.33 | 1.35 |
| TSM-140 | | | | | | |
| 32164-003-46 Ca EP-9174 | 94.67 | 1.90 | 0.83 | 0.86 | 1.43 | 1.44 |
| 32164-027-44 Ca exchanged and | 94.59 | 2.47 | 0.75 | 0.75 | 1.28 | 1.27 |
| fired* | | | | | | |
| 32164-027-46 Ca exchanged and | 94.77 | 1.71 | 0.43 | 0.43 | 0.81 | 0.80 |
| fired* | | | | | | |
| 07384-24-16 TX-764 fired* | 97.14 | 1.68 | 0.21 | 0.24 | 0.74 | 0.76 |

*Fired at 496° C. for 1 hour in house air (dry)

Equilibrium was apparently achieved after 1 hour with the 6 torr data points because of the very small change in weight between the 1 hour and 1.9 hour readings compared to the 1 hour reading. For the second point (46 torr), the first reading was taken at 30 minutes and the second at 1 hour. Equilibrium is achieved at this shorter time indicating that the carbon monoxide rate of adsorption is rapid.

These samples were then subjected to vacuum at room temperature for 1.5 hours and most of the CO desorbed (Table 7) then the samples were vacuum activated overnight at 350° C. The next day, they were tested for ethylene adsorption at 750 torr and 22° C. For ethylene exclusion (less is better) the following was found (Table 7):

CaEP1974<CaTSM-140*=CaTX-764=TX-764<TSM-140<CaTSM-140<<CaTX-764<TX-764

TABLE 7
McBain Result of CO Desorption and Ethylene Adsorption

| | Sample # | | | | |
|---|---|---|---|---|---|
| | Loading | Loading | Loading | Loading | Loading |
| | WT % | WT % | WT % | WT % | WT % |
| | Ads. Press. (VG), Torr | | | | |
| | 1.8E−02 | 8.0E−03 | | | |
| | Ads. Press. (PV), Torr | | | | |
| | | | 749 | 749 | 749 |
| | Ads. Time, Hr | | | | |
| | Evac. | | | | |
| | @ 22 | Evac. @ | | | |
| | C. for | 350 C. | Ethylene | Ethylene | Ethylene |
| Sample # | 1.5 hr | overnight | @ 1.5 hrs | @ 5.0 hrs | @ 23.5 hrs |
| 07384-24-9 TSM-140 | 0.00 | −0.09 | 0.31 | 0.41 | 0.76 |
| 07384-24-16 TX-764 | 0.02 | −0.08 | 0.34 | 0.78 | 1.76 |
| 32164-027-44 Ca TX-764 | 0.33 | −0.10 | 0.33 | 0.69 | 1.44 |
| 32164-027-46 Ca TSM-140 | 0.27 | −0.02 | 0.44 | 0.59 | 0.95 |
| 32164-003-46 Ca EP-9174 | 0.62 | 0.00 | 0.00 | 0.04 | 0.24 |
| 32164-027-44 Ca TX-764 fired* | 0.37 | 0.07 | 0.13 | 0.23 | 0.56 |
| 32164-027-46 Ca TSM-140 fired* | 0.22 | 0.13 | 0.37 | 0.42 | 0.51 |
| 07384-24-16 fired* | 0.11 | 0.12 | 0.16 | 0.30 | 0.61 |

*Fired at 496° C. for 1 hour in house air (dry).

After the ethylene uptake, the McBain was evacuated and flooded with helium to keep the samples dry over the weekend. Then ethylene was introduced again at 750 torr. Some of the samples lost weight at the first CO adsorption point which was at 6 torr and 2 hours. Therefore, it is not clear as to the CO rate or final CO loading on these samples. The CO pressure was increased to 46 torr and the difference between 6 and 46 could be attributed to just CO uptake but there is no way to know how much ethylene desorbed over this time. An approximation can be made if we just look at the difference between 6 and 46 torr and assume that any ethylene desorption would be negligible. If so, then the highest CO values would belong to the best candidates when ethylene is present. The order of CO capacity under these conditions is as follows (Table 8):

Ca TX-764*=Ca EP-9174>Ca TSM-140>Ca TX-764=TX-764*>Ca TSM-140*=TSM-140>TX-764

TABLE 8
McBain Result of CO Adsorption after Ethylene Preloading

| | Sample # | | | |
|---|---|---|---|---|
| | Loading | Loading | Loading | Loading |
| | WT % | WT % | WT % | WT % |
| | Ads. Press. (VG), Torr | | | |
| | Ads. Press. (PV), Torr | | | |
| | 750 | 6 | 46 | |
| | Ads. Time, Hr | | | |
| | | | | CO delta |
| | Ethylene | CO @ | CO @ | (46 − 6 |
| Sample # | @ 1.1 hrs | 2.0 hrs | 2.5 hrs | torr) |
| 07384-24-9 TSM-140 | 0.77 | 0.70 | 1.15 | 0.45 |
| 07384-24-16 TX-764 | 1.39 | 1.46 | 1.79 | 0.33 |
| 32164-027-44 Ca TX-764 | 1.25 | 1.35 | 1.89 | 0.54 |
| 32164-027-46 Ca TSM-140 | 0.92 | 1.10 | 1.77 | 0.67 |
| 32164-003-46 Ca EP-9174 | 0.26 | 0.55 | 1.31 | 0.76 |
| 32164-027-44 Ca TX-764 | 0.53 | 0.79 | 1.56 | 0.77 |
| fired* | | | | |
| 32164-027-46 Ca TSM-140 | 0.49 | 0.54 | 1.02 | 0.48 |
| fired* | | | | |
| 07384-24-16 fired* | 0.51 | 0.66 | 1.18 | 0.52 |

The samples were again activated overnight at 350° C. under vacuum and ethane was introduced to the system. It appears that the ethane content of typical catalytic reformer net hydrogen gas is significantly higher than the trace levels of ethylene that may be found. Therefore, ethane exclusion is much more important. The samples were exposed to ethane at 750 torr and 22° C. for 2 hours and overnight. The data is presented in Table 9. The material with the lowest ethane uptake should be the best. The order of ethane uptake found was:

Ca EP-9174<Ca TSM-140*<TX-764*=Ca TX-764*<TSM-140=Ca TSM-140<Ca TX-764<TX-764

TABLE 9
McBain Result of Ethane Uptake on Calcium Clinos

| | Sample # | | |
|---|---|---|---|
| | Loading | Loading | Loading |
| | WT % | WT % | WT % |
| | Ads. Press. (VG), Torr | | |
| | 1.7E−02 | | |
| | Ads. Press. (PV), Torr | | |
| | | 749 | 746 |
| | Ads. Time, Hr | | |
| | Evac. @ | | |
| | 350 C., | Ethane | Ethane |
| Sample # | 16 hr | @ 2.0 hrs | @ 23 hrs |
| 07384-24-9 TSM-140 | −0.11 | 0.22 | 0.51 |
| 07384-24-16 TX-764 | −0.08 | 0.19 | 0.91 |
| 32164-027-44 Ca TX-764 | −0.05 | 0.18 | 0.76 |
| 32164-027-46 Ca TSM-140 | −0.05 | 0.30 | 0.55 |
| 32164-003-46 Ca EP-9174 | −0.01 | −0.05 | 0.15 |
| 32164-027-44 Ca TX-764 fired* | 0.12 | 0.12 | 0.39 |
| 32164-027-46 Ca TSM-140 fired* | −0.02 | 0.22 | 0.31 |
| 07384-24-16 fired* | 0.06 | 0.14 | 0.38 |

*Fired at 496° C. for 1 hour in house air (dry)

It appears that firing of the exchanged clino can have an advantageous effect on the performance (most notably in the hydrocarbon exclusion) of clino regardless of the cation type. Based upon the data, it appears that the calcium exchanged EP-9174 (of the sodium exchanged clino EP-9174), although chemically not much different than the calcium exchanged raw ore, is still the best candidate for the Platformer H2 net gas application.

1. A process for separating a minor proportion of carbon monoxide from a hydrocarbon or hydrogen containing stream, which process comprises contacting the carbon monoxide-containing mixture with an adsorbent having an effective pore size and shape that excludes hydrocarbon molecules and is large enough to adsorb carbon monoxide molecules.

2. The process of claim 1 wherein said adsorbent is a natural clinoptilolite that has been subjected to ion-exchange with at least one metal cation of the group consisting of lithium, sodium, potassium, calcium, magnesium, and barium cations, thereby causing the carbon monoxide to be selectively adsorbed into the clinoptilolite.

3. The process of claim 2 wherein the metal cation is calcium.

4. The process of claim 2 wherein the metal cation is a mixture of calcium and sodium.

5. The process of claim 2 wherein the metal cation is barium.

6. The process of claim 1 wherein said natural clinoptilolite is fired at a temperature of about 300 to 650° C. for a suitable period of time.

7. The process of claim 1 wherein the carbon monoxide content of the hydrogen or hydrocarbon containing stream is not greater than about one percent by weight.

8. The process of claim 1 wherein said hydrogen containing stream is produced from a catalytic reforming unit.

9. The process of claim 1 wherein said adsorbent is used for purification of make-up hydrogen to a paraffin or olefin isomerization unit.

10. The process of claim 1 wherein said adsorbent is used for purification of olefins in an olefin production process.

11. The process of claim 1 wherein said hydrogen containing stream is produced from a steam reforming reaction.

12. The process of claim 1 further comprising regeneration of said adsorbent.

13. The process of claim 1 further comprising removing carbon dioxide.

14. A process for the production of high purity hydrogen from a catalytic reformer which process comprises the steps including:
(a) passing at least a portion of a hydrogen gas stream produced in the catalytic reformer and comprising carbon monoxide to a adsorbent bed containing an adsorbent having an effective pore size and shape that excludes hydrocarbon molecules and is large enough to adsorb carbon monoxide molecules and
(b) passing at least a portion of the hydrogen gas stream having a reduced concentration of carbon monoxide to a catalytic hydrocarbon conversion process requiring hydrogen containing low levels of carbon monoxide.

15. The process of claim 14 wherein said adsorbent is a natural clinoptilolite that has been subjected to ion-exchange with at least one metal cation of the group consisting of lithium, sodium, potassium, calcium, magnesium, and barium cations, thereby causing the carbon monoxide to be selectively adsorbed into the clinoptilolite.
